(19) United States
(12) Patent Application Publication
(10) Pub. No.: US 2025/0032539 A1
(43) Pub. Date: Jan. 30, 2025

(54) PHARMACEUTICAL COMPOSITION FOR PREVENTING AND TREATING BRAIN INJURY ASSOCIATED WITH FERROPTOSIS AFTER INTRACEREBRAL HEMORRHAGE AND METHOD THEREOF

(71) Applicant: THE FIRST HOSPITAL AFFILIATED TO PLA ARMY MEDICAL UNIVERSITY, CHONGQING (CN)

(72) Inventor: RONG HU, CHONGQING (CN)

(21) Appl. No.: 18/810,495

(22) Filed: Aug. 20, 2024

Related U.S. Application Data
(63) Continuation of application No. PCT/CN2023/133117, filed on Nov. 22, 2023.

(30) Foreign Application Priority Data
Feb. 22, 2023 (CN) 202310165091.3

(51) Int. Cl. A61K 35/16; A61P 25/28

(52) U.S. Cl. CPC A61K 35/16; A61P 25/28

(57) ABSTRACT

A method for preventing and treating brain injury associated with ferroptosis after intracerebral hemorrhage includes a step of administering human plasma exosomes to a subject. The human plasma exosome is derived from plasma of healthy young people. A pharmaceutical composition for preventing and treating brain injury associated with ferroptosis after intracerebral hemorrhage includes the human plasma exosomes and pharmaceutical excipients. The human plasma exosomes reduce brain injury by inhibiting ferroptosis, and significantly enhance behavioral recovery of ICH patients. Moreover, the human plasma exosomes have no obvious side effects, nor cause organ toxicity, and are well tolerated and safe.

### TECHNICAL FIELD

The present disclosure relates to a field of biomedicine, and in particular to a pharmaceutical composition for preventing and treating brain injury associated with ferroptosis after intracerebral hemorrhage and a method thereof.

### BACKGROUND

Intracerebral hemorrhage (ICH) is a life-threatening disease, accounting for 15% to 20% of strokes, and most survivors suffer from lifelong disability. A pathological basis for poor prognosis of the ICH is primary brain injury (PBI) and secondary brain injury (SBI). The SBI is characterized by a series of complex pathological processes such as oxidative stress, inflammation, and apoptosis. Ferroptosis is a newly discovered cell autophagic death mode that is different from other cell death modes. The ferroptosis causes a variety of brain diseases, including neurodegenerative diseases, and the ferroptosis is characterized by reactive oxygen species (ROS) accumulation and lipid peroxidation. Especially in the ICH, ferrous iron produced by hematoma degradation contributes to an accumulation of the ROS produced by a Fenton reaction, and the ferrous iron also acts as a cofactor for the lipid peroxidation. Therefore, after the ICH, the ROS accumulation and iron deposition easily induce the ferroptosis. In fact, recent studies also suggests that the ferroptosis of cells around the hematoma plays a vital role in the SBI after the ICH. Therefore, strategies to specifically inhibit ferroptosis after the ICH is promising.

Exosomes are small vesicles with a diameter of 50-150 nm, which have an ability to transfer nutrients and RNA sequences between different types of cells. Studies have shown that exosomes derived from mesenchymal stem cells, exosomes derived from neural stem cells, exosomes derived from astrocytes, and exosomes derived from microglia are able to protect neurons from various injuries such as the ICH through different pathways. However, it is not clear whether the exosomes can protect the neurons from ferroptosis.

The exosomes exist in a circulation, and a production thereof in plasma (or serum) is much higher than a production of the exosomes secreted by a single cell type. Previously, convalescent plasma therapy has become an attractive treatment strategy for infectious diseases and immune diseases. Conventionally, plasma therapy is believed to provide antibodies and nutrients to patients. In recent years, the exosomes derived from the plasma are found to have a variety of therapeutic effects. In particular, it has been reported that the exosomes derived from the plasma of healthy young people have good application prospects in treatment of inflammation and COVID 19. However, an effect of the exosomes derived from the plasma of healthy young people on the ICH and potential thereof are still unclear. Therefore, potential of studying and developing pharmaceutical composition derived from the exosomes from the healthy young people for preventing and treating the ICH has important application and socioeconomic value.

### SUMMARY

The present disclosure provides a pharmaceutical composition for preventing and treating brain injury associated with ferroptosis after intracerebral hemorrhage and a method thereof. The pharmaceutical composition comprises human plasma exosomes, and the human plasma exosomes are derived from plasma of healthy young people and are therefore referred to as “human plasma exosomes” in the present disclosure.

The method for preventing and treating brain injury associated with ferroptosis after intracerebral hemorrhage (ICH) comprises administering human plasma exosomes to a subject. The human plasma exosome is derived from plasma of healthy young people.

Optionally, the healthy young people are between 18 and 25 years old.

Optionally, the human plasma exosomes are extracted and separated from the plasma of the healthy young people.

Optionally, a dose of human plasma exosomes administered to a mouse is 40-60 μg.

The present disclosure provides the pharmaceutical composition for preventing and treating brain injury associated with ferroptosis after ICH. The pharmaceutical composition comprises human plasma exosomes and pharmaceutical excipients. The human plasma exosome is derived from plasma of healthy young people.

Optionally, the healthy young people are between 18 and 25 years old.

Optionally, the pharmaceutical composition is in a form of an injection.

The injection is an injection solution or a lyophilized powder injection, and the pharmaceutical excipients are conventional injection excipients in the prior art, such as mannitol, lactose, water for injection, physiological saline, glucose, etc. The injection is prepared by a conventional methods in the prior art. For example, the injection in obtained by mixing the human plasma exosomes with the pharmaceutical excipients including the water for injection, sterilizing, filling, and optionally freeze-drying.

The human plasma exosomes of the present disclosure are allowed to be taken up by neurons and microglia, and are capable of reducing the ferroptosis of cells after the ICH, thereby preventing the brain injury and accelerating behavioral recovery of ICH patients.

The human plasma exosomes of the present disclosure effectively alleviate a ferroptosis phenomenon after the ICH in mice and promote the behavioral recovery of the ICH patients.

### BRIEF DESCRIPTION OF DRAWINGS

FIG. 1A is a transmission electron microscopy image of the human plasma exosomes human from healthy young people.

FIG. 1B is a Zeta potential image of the human plasma exosomes human plasma exosomes from healthy young people.

FIG. 1C is a schematic diagram of an expression of exosome markers CD63, CD9, CD81, and TSG101 in the human plasma exosomes from healthy young people.

FIG. 1D is a schematic diagram showing a particle size distribution of the human plasma exosomes from healthy young people.

FIG. 2A is a schematic diagram showing an ICH autologous blood injection model and coronal positions of the lateral ventricular injection in a mouse brain atlas.

FIG. 2B is a schematic diagram showing the ICH autologous blood injection models and actual positions of the lateral ventricular injection.

FIG. 3A is a schematic diagram showing an in vivo distribution of DiD-labeled human plasma exosomes in brains of three sham-operated mice and three ICH mice through confocal imaging.

FIG. 3B is a schematic diagram showing that the DiD-labeled human plasma exosomes are taken up by neurons and microglia through confocal imaging, but not by astrocytes.

FIG. 4A is a graph showing results of an open field experiment in the ICH mice after injection of different doses of the human plasma exosomes derived from healthy young people.

FIG. 4B is a graph showing results of behavior scores of the ICH mice after injection of different doses of the human plasma exosomes derived from the healthy young people.

FIG. 4C is a graph showing the times of contralateral limbs of the ICH mice slipped when walking on a beam after being injected with different doses of the human plasma exosomes derived from the healthy young people.

FIG. 4D is a graph showing results of an open field experiment in ICH mice after injection of different doses of human plasma exosomes derived from healthy elderly people.

FIG. 4E is a graph showing results of behavior scores of the ICH mice after injection of different doses of the human plasma exosomes derived from the healthy elderly people.

FIG. 4F is a graph showing the times of THE contralateral limbs of the ICH mice slipped when walking on THE beam after being injected with different doses of the human plasma exosomes derived from the healthy elderly people.

FIG. 5A is an image showing gross injury of brains of the ICH mice in a Sham group, an ICH group, and an ICH+EXO group observed by the HE staining.

FIG. 5B is a representative micrograph of the Nissl staining in different groups.

FIG. 5C is a bar graph showing quantification of the Nissl-positive neurons in the experiment.

FIG. 5D is a schematic diagram showing representative immunofluorescence images of the ferroptosis marker GPX4 around the hematoma in different groups.

FIG. 5E is a bar graph showing a result of a quantitative analysis of GPX4 staining.

FIG. 5F is a schematic diagram showing transmission electron microscopy images showing changes in mitochondria in different groups.

FIG. 6A is a schematic diagram showing detection results of SLC7A11 and GPX4 around hematoma in different groups through a Western Blot method.

FIG. 6B is a bar graph of analysis results for quantitative analysis of SLC7A11.

FIG. 6C is a bar graph of analysis results for quantitative analysis of GPX4.

FIG. 6D is a bar graph of quantitative analysis of malondialdehyde (MDA) around the hematoma of different groups.

FIG. 6E is a bar graph of results of quantitative analysis of GSH around the hematoma of different groups.

FIG. 6F is a bar graph of detection results of the expression levels of SLC7A11 and GPX4 m RNA around the hematoma of different groups by using a real-time quantitative polymerase chain reaction (qRT-PCR).

FIG. 6G is a bar graph of detection results of the expression levels of GPX4 m RNA around the hematoma of different groups by using a real-time qRT-PCR.

The drawings are shown in colors for illustrative purposes only and the colors thereof form no part of the claimed design.

### DETAILED DESCRIPTION

Following embodiments are used to further understand and illustrate the essence of the present disclosure, but do not limit the scope of the present disclosure in any way.

Experimental animals C57BL/6J in the embodiments are purchased from Hunan Enswell Experimental Animal Technology Co., Ltd.

Plasma of healthy young people is collected with approval of the Ethics Committee of Southwest Hospital of Army Medical University.

### Embodiment 1 (Extraction of Human Plasma Exosomes)

The embodiment provides a method for extracting human plasma exosomes. The method comprises steps A-H.

Step A: collecting peripheral blood of healthy young people and healthy elderly people by using an ethylene diamine tetraacetic acid (EDTA) anticoagulant collection tubes, and standing for 2 h, where the healthy young people are between 18 and 25 years old.

Step B: centrifuging the peripheral blood at 2000 g for 15 minutes at 4° C. in a refrigerated high-speed centrifuge, and taking upper plasma therein as a primary plasma.

Step C: centrifuging the primary plasma at 2000 g for 15 minutes at 4° C. in the refrigerated high-speed centrifuge, taking upper plasma thereof as secondary plasma, dividing and packaging the secondary plasma, and storing packaged secondary plasma in a refrigerator at −80° C. to obtain packaged frozen plasma;

Step D: taking out the packaged frozen plasma, thawing the packaged frozen plasma in a water bath at 25-37° C., and place packaged thawed plasma on ice after thawing.

Step E: centrifuging the packaged thawed plasma at 2000 g for 10 minutes at 4° C. in the refrigerated high-speed centrifuge, and taking supernatant therein as primary supernatant.

Step F: centrifuging the primary supernatant at 12000 g for 30 minutes at 4° C. in the refrigerated high-speed centrifuge, and taking supernatant therein as secondary supernatant;

Step G: filtering the secondary supernatant with a 0.22 μm microporous filter membrane, then slowly pouring the secondary supernatant into a special ultracentrifuge tube, and then diluting the secondary supernatant with filtered sterile phosphate buffered saline (PBS) to obtain diluted supernatant.

Step H: centrifuging the diluted supernatan at 120,000 g for 120 minutes at 4° C. in a refrigerated ultracentrifuge, slowly aspirating supernatant therein as final supernatant. resuspending precipitate with 100 μL sterile PBS to obtain the human plasma exosomes.

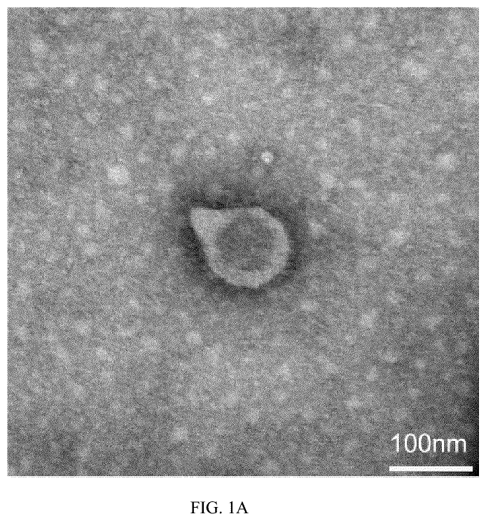

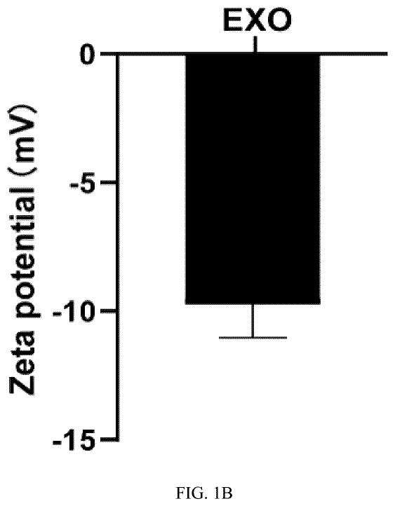

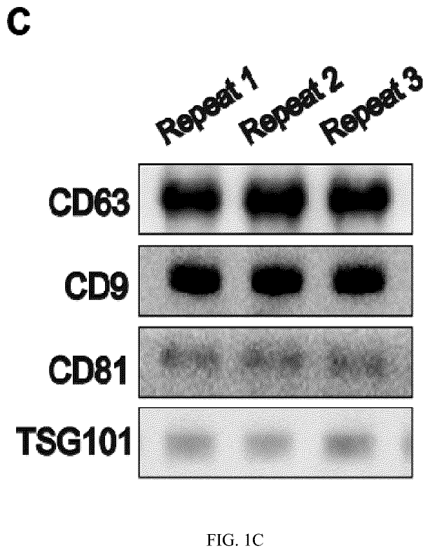

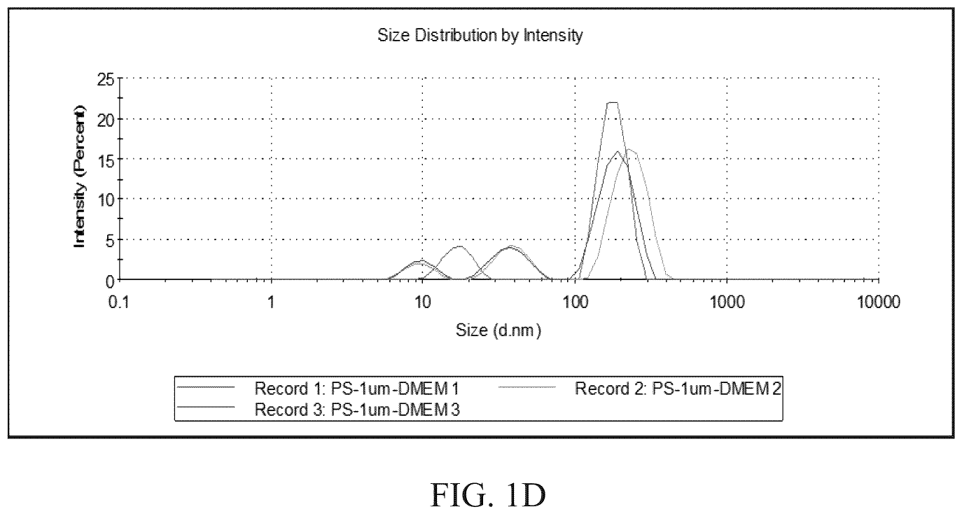

Transmission electron microscopy (TEM), Nanoparticle Tracking Analysis (NTA), zeta potential measurement, and exosome marker western blot are used to identify the human plasma exosomes. Results thereof are shown in FIGS. 1A-1D.

### Embodiment 2 (Labeling of the Human Plasma Exosomes of the Healthy Young People)

Fluorescently labeled human plasma exosomes are synthesized by using a DiD kit. Specifically, DiD dye is added to an exosome resuspension and incubated for 10 minutes to stain the human plasma exosomes. Then, the DiD dye in the exosome resuspension is removed by centrifugation at 100,000 g for 2 h at 4° C. to obtain precipitate. Then, the precipitate was washed with PBS to obtain dye-stained exosomes.

### Embodiment 3 (Construction of ICH Mouse Model)

Experimental animals C57BL/6J in the embodiment are purchased from Hunan Enswell Experimental Animal Technology Co., Ltd., and are approved by the Ethics Committee of Southwest Hospital of Army Medical University.

An autologous blood injection method is provided, and the autologous blood injection method is specifically illustrated as follow.

First, first mice are anesthetized with 2% isoflurane/air mixed gas, and the first mice are fixed on a stereotaxic instrument. Then, an anterior bregma and an posterior bregma of each of the first mice are exposed, and a hole is drilled 0.8 mm in front of the anterior bregma and 2 mm away from the midline of each of the first mice. Then. a tail of each of the first mice is disinfected with 75% alcohol, and 25 μL of autologous blood is collected from the tail of each of the first mice by a microsyringe, and the autologous blood is slowly injected into basal ganglia of a corresponding first mouse to a depth of 3.5 mm at a speed of 2 μL/min.

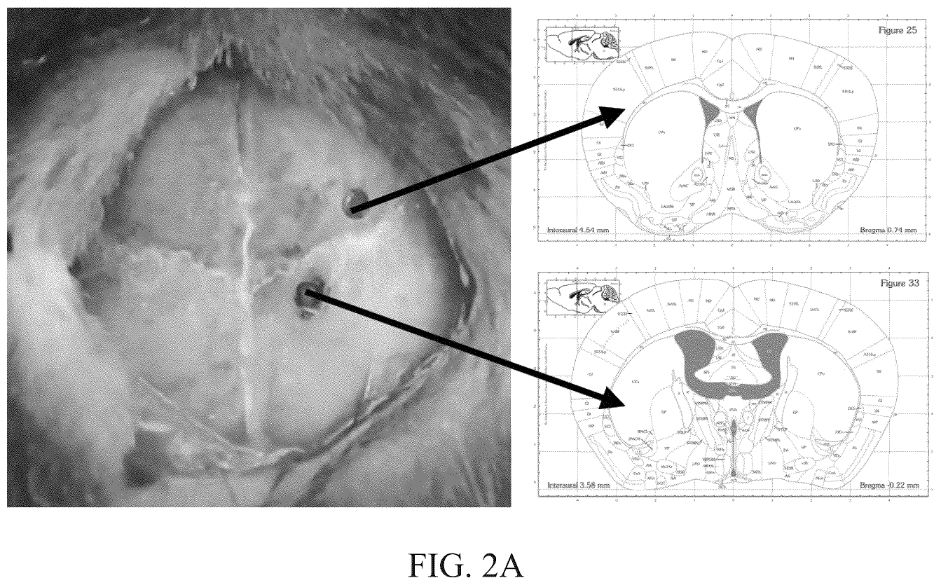

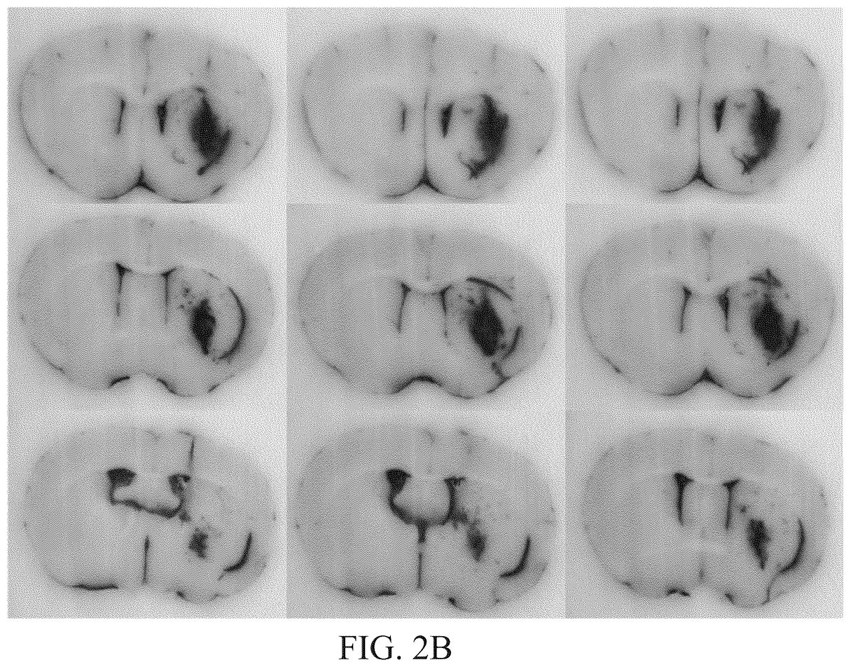

For second mice in a Sham group, same operations are performed, but the autologous blood is replaced with saline of 25 25 μL. During the operations, a body temperature of the second mice is maintained at 37° C.±0.3° C. by a constant temperature heating plate until the second mice woke up. The operations are performed by skilled researchers to minimize pain or distress of the second mice during the operations. After surgery, the second mice have free access to food and water under a constant light cycle (12 hours of light/darkness). The ICH position of the mice and successful models are shown in FIGS. 2A-2B.

### Embodiment 4 (Distribution and Absorption of Human Plasma Exosomes in Brains of ICH Mice)

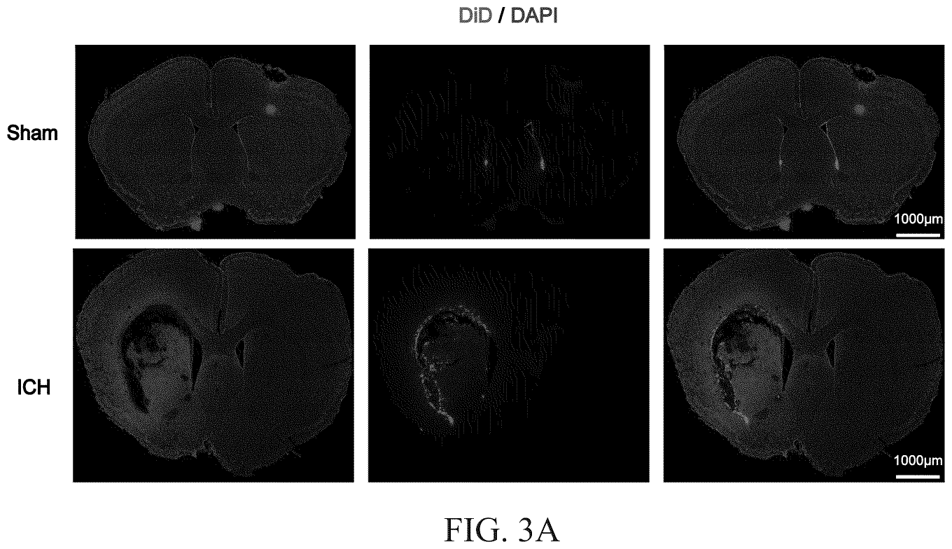

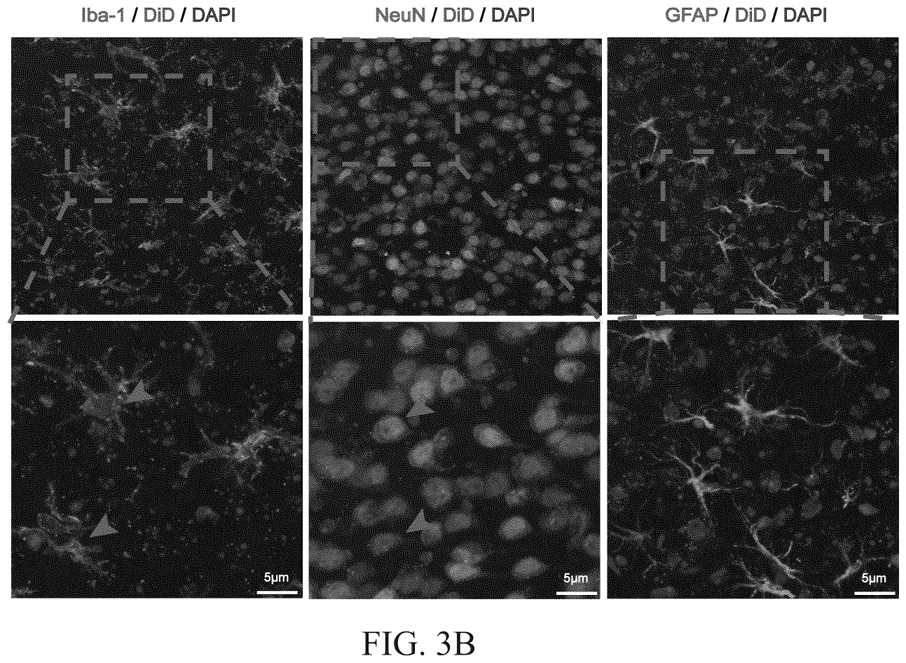

The dye-stained exosomes from healthy young human blood is injected into the ICH mice. An injection site is located in a lateral ventricle of a hemisphere of a brain of each of the ICH mice. The ICH mice are injected once a day for the first three days after ICH. On the third day, brain images of the ICH mice are collected to observe a distribution of the dye-stained exosomes, and results thereof are shown in FIGS. 3A-3B.

An injection process comprises steps (1)-(3).

The step (1) comprises collecting the dye-stained exosomes, performing concentration measurement by using a BCA kit, accurately determining a volume of the dye-stained exosomes, and placing the dye-stained exosomes in the refrigerator at −80° C. for freezing;

The step (2) comprises melting the dye-stained exosomes at room temperature, then respectively injecting 20 μg, 40 μg, and 60 μg of the dye-stained exosomes into the lateral ventricle of the hemisphere of the brain of each of the ICH mice and injecting same volume of the PBS into the ICH mice in a control group.

The step (3) comprises injecting the ICH mice once a day for the first three days after ICH, injecting the ICH mice once again on the seventh day. Namely, each of the ICH mice is injected for four times.

### Embodiment 5 (Comparison of Activity Between Human Plasma Exosomes from Healthy Young People and Human Plasma Exosomes from Healthy Elderly People)

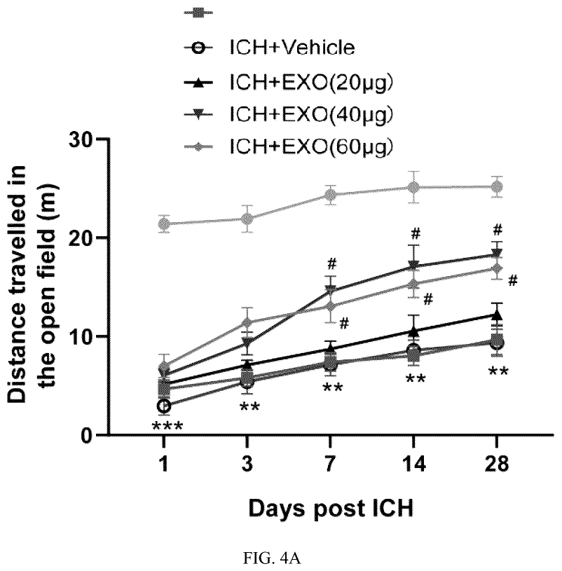

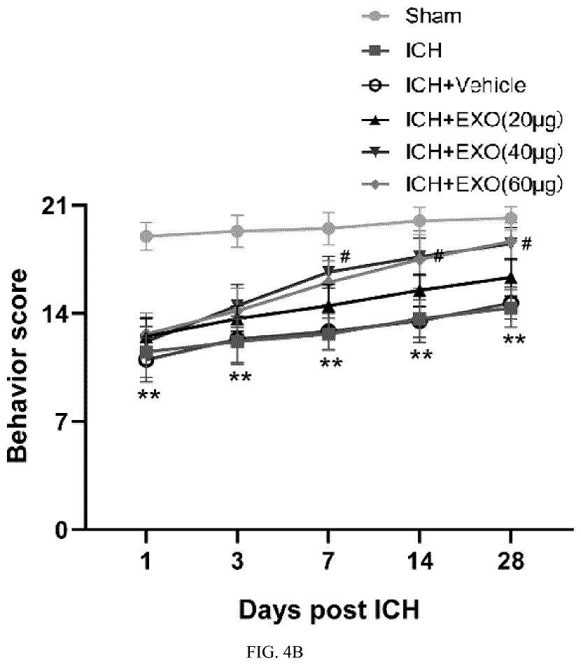

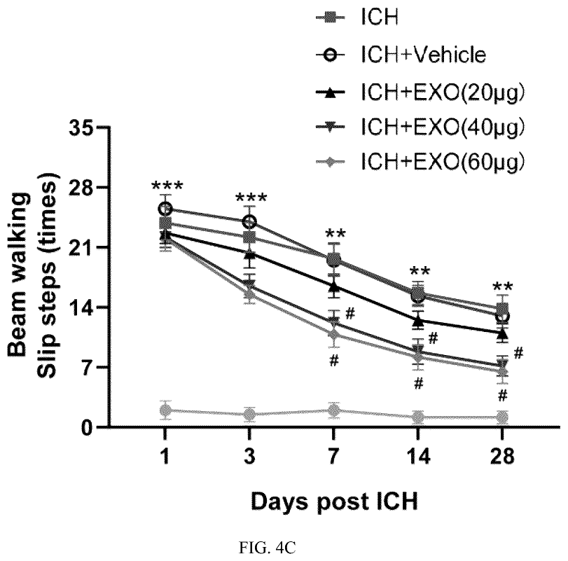

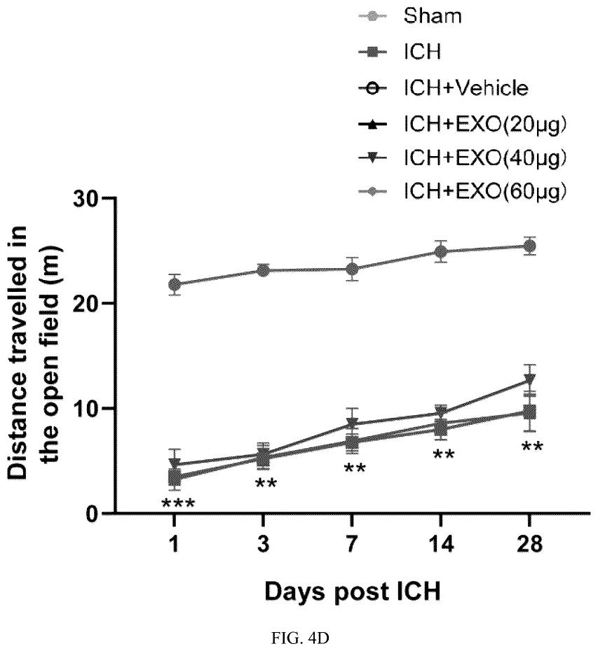

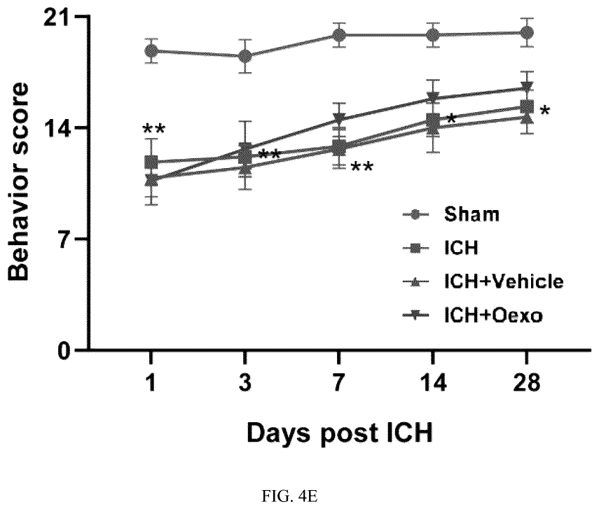

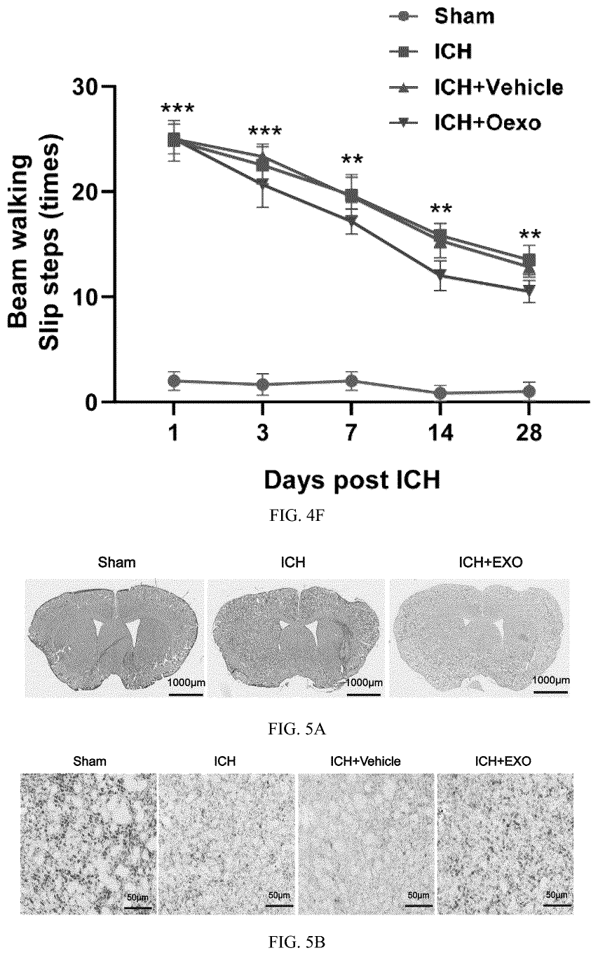

After extracting the human plasma exosomes from healthy young people and healthy elderly people, the human plasma exosomes are dyed and injected into the ICH mice according to the injection process of Embodiment 4, and behaviors of the ICH mice in the ICH group and the ICH mice the exosome injection group (hereinafter the ICH+EXO group) is tested on the 1^{st} day, 2^{nd} day, 3^{rd} day, 7^{th} day, 14^{th} day, and 28^{th} day, including an open field test, a beam-walking test, and a modified Garcia score, and results thereof are shown in FIGS. 4A-4F-.

FIG. 4A-4F are dose-response curves of the human plasma exosomes derived from healthy young people and the human plasma exosomes derived from healthy elderly people in behavioral recovery of the ICH mice. n=6. n represent the number of the ICH mice.

In the figures, ** P<0.01, *** P<0.001 represent the ICH group vs. the Sham group. #P<0.05 represents the ICH+EXO group vs. the ICH group. FIGS. 4A-4C show motor functions of the ICH mice respectively injected with 20 μg, 40 μg, and 60 μg of the human plasma exosomes from the healthy young people into the lateral ventricle thereof. FIGS. 4A-4C further specifically show the motor functions of the ICH mice observed 1 day, 3 days, 7 days, 14 days, and 28 days after injection of different doses of the human plasma exosomes from the healthy young people. FIG. 4A shows a result of the open field test. The open field test records a total distance that each of the ICH mice move within a specified time. The longer the total distance, the better the behavioral recovery of the ICH mice. Compared with the ICH mice injected with 20 μg and 60 μg of the human plasma exosomes from the healthy young people, the ICH mice injected with 40 μg of the human plasma exosomes from the healthy young people performed best in the open field test and moved the farthest distance in 28 days. FIG. 4B shows the Modified Garcia scores of each group of the ICH mice. The higher the score of the ICH mice, the better the behavioral recovery of the ICH mice. On the 28th day, compared with the ICH mice injected with 20 μg of the human plasma exosomes from the healthy young people, the ICH mice injected with 40 μg and 60 μg of the human plasma exosomes from the healthy young people scored higher.

FIG. 4C shows a result of a beam walking test, which records the number of times the contralateral limbs of the ICH mice slipped when walking on a beam. The results of the beam walking test reflect fine movements of the ICH mice. The fewer the number of slips, the better the coordination of the ICH mice. On the 28th day, compared with the ICH mice injected with 20 μg of the human plasma exosomes from the healthy young people, the ICH mice injected with 40 μg and 60 μg of the human plasma exosomes from the healthy young people have fewer limb slips when walking on the beam.

In summary, both 40 μg and 60 μg injections of the human plasma exosomes from the healthy young people are optimal doses, especially 40 μg injection of the human plasma exosomes from the healthy young people, which performs best in all aspects. Therefore, 40 μg of the human plasma exosomes from the healthy young people is selected as the dose used in subsequent tests. FIGS. 4D-4E show a motor function of the ICH mice injected with 40 μg, of the human plasma exosomes from the healthy elderly people into the lateral ventricle thereof after 1 day, 3 days, 7 days, 14 days, and 28 days. FIG. 4D shows the results of the open field test, FIG. 4E shows behavioral scores, and FIG. 4F shows the results of the beam walking test. To sum up, there is no significant difference in the behavioral performance of the ICH mice after injection of the human plasma exosomes from the healthy elderly people with the ICH mice in the ICH group. Through these comparisons, it is found that the human plasma exosomes from the healthy young people promote the recovery of the motor function of the ICH mice, while the human plasma exosomes from the healthy elderly people have no effect. The results are amazing. The human plasma exosomes from the healthy elderly people have no therapeutic effect on the brain injury associated with ferroptosis after ICH while the human plasma exosomes from the healthy young people, especially from those aged 20-25, show efficacy on brain injury associated with ferroptosis after ICH. Further, the dose of 40-60 μg of the human plasma exosomes from the healthy young people shows significant therapeutic effects on the brain injury associated with ferroptosis after ICH.

Therefore, 40 μg of the human plasma exosomes from the healthy young people are selected as experimental materials for the following further experiments.

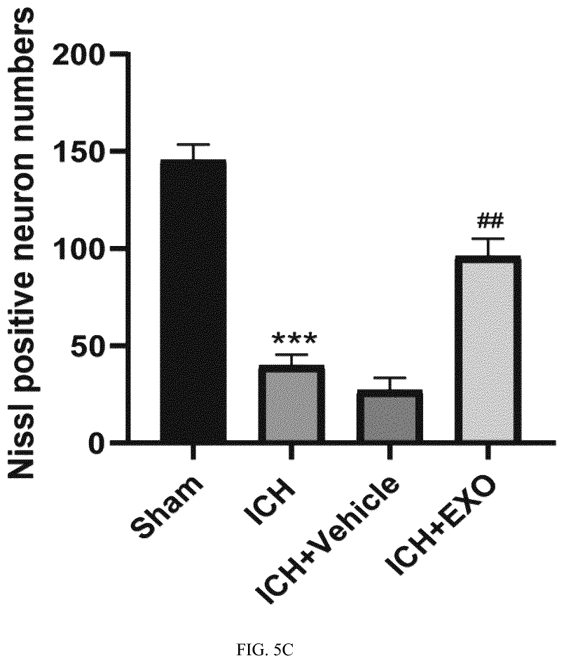

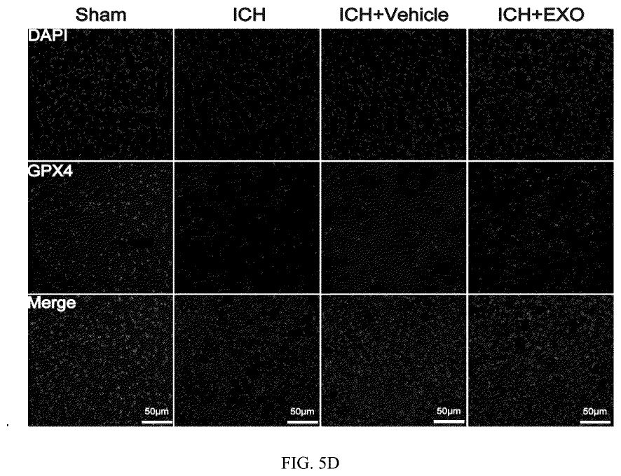

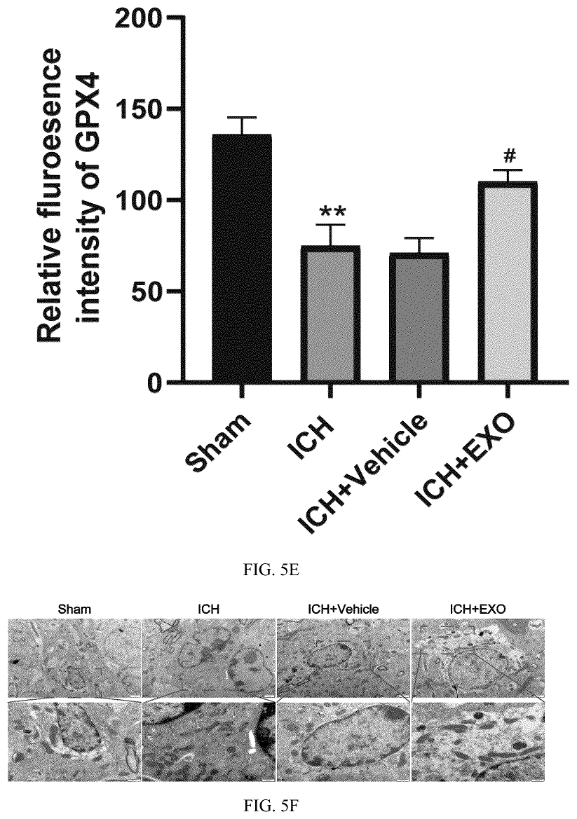

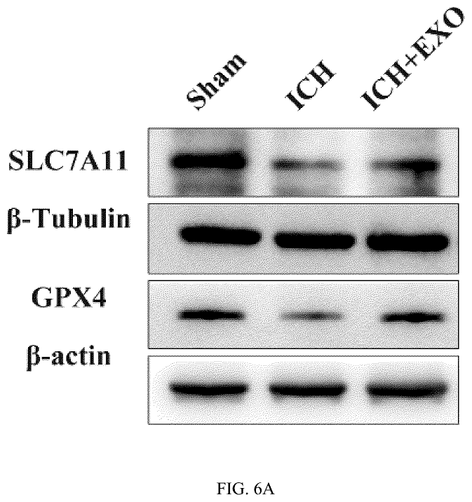

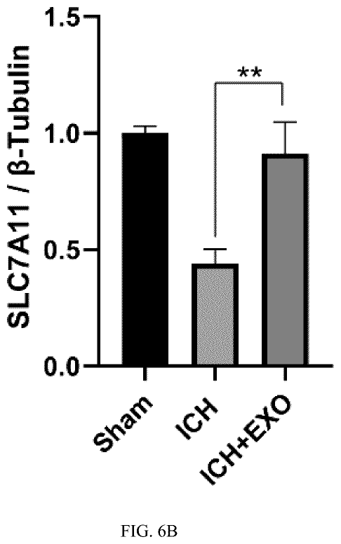

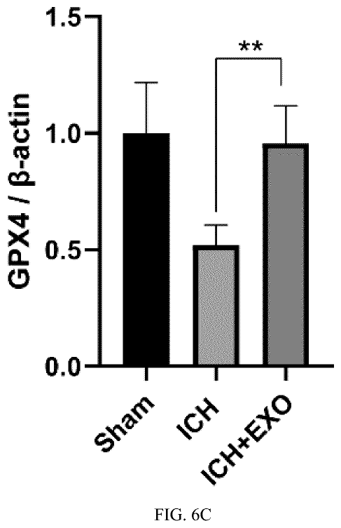

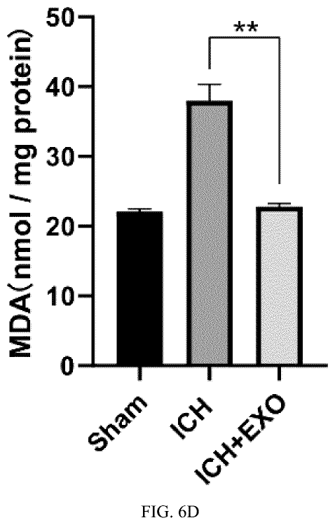

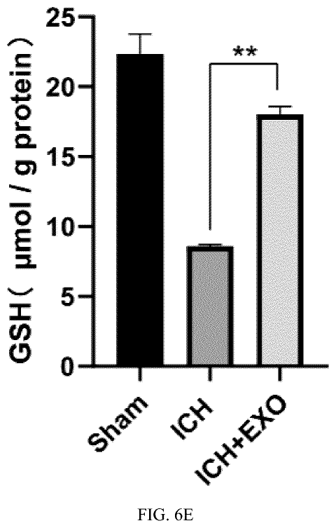

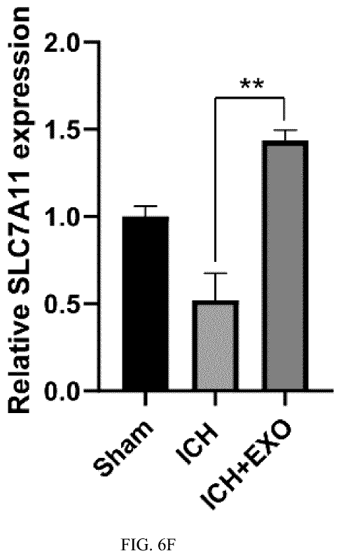

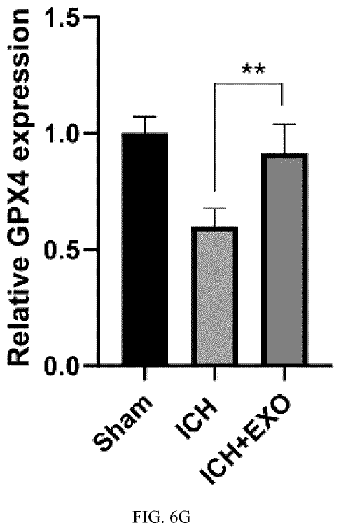

On the seventh day, brain images of the ICH mice injected with the human plasma exosomes from the healthy young people are collected to detect following indicators:
(1) Observing brain injury and neuronal injury after ICH through HE staining and Nissl staining;
(2) Observing an expression of ferroptosis marker GPX4 around the hematoma after ICH through immunofluorescence staining;
(3) Detecting the mitochondrial morphology around the hematoma of the ICH mice through a transmission electron microscope, and results thereof are shown in FIG. 5;
(4) Detecting changes in an gene expression of ferroptosis markers GPX4 and SLC7A11 through a Western blot method;
(5) Detecting the mRNA expression of ferroptosis markers GPX4 and SLC7A11 through a real-time quantitative polymerase chain reaction (qPCR) method; and
(6) Detecting expression levels of malondialdehyde (MDA) and glutathione (GSH), and the results thereof are shown in FIGS. 6A-6F.

FIGS. 5A-5F show an effect of the human plasma exosomes from the healthy young people on reducing brain injury and ferroptosis after ICH, where n=3, ** P<0.01, *** P<0.001 represent ICH group vs. Sham group; #P<0.05, ##P<0.01 represent ICH group+EXO group vs. ICH group. FIG. 5A shows gross injury of the brains of the ICH mice in the Sham group, the ICH group, and the ICH+EXO group observed by the HE staining. FIG. 5B is a representative micrograph of the Nissl staining in different groups. FIG. 5C shows quantification of the Nissl-positive neurons in the experiment. FIG. 5D shows representative immunofluorescence images of the ferroptosis marker GPX4 around the hematoma in different groups. FIG. 5E show a result of a quantitative analysis of GPX4 staining. FIG. 5F shows transmission electron microscopy images showing changes in mitochondria in different groups. To sum up, the results of the ICH+EXO group shows significant differences in mitochondrial changes, GPX4 expression density around the hematoma, brain injury, and positive neuron injury compared with the ICH group and an ICH blank groups. The results of the ICH+EXO group are close to or equivalent to that of the Sham group. The results of the ICH+EXO group show significant curative effect and are statistically significant.

FIGS. 6A-6G show an effect of the human plasma exosomes from the healthy young people on counteracting ferroptosis caused by ICH through an SLC7A11/GPX4 pathway, where n=6, ** P<0.01 represents the ICH+EXO group VS the ICH group. FIG. 6A shows detection results of SLC7A11 and GPX4 around hematoma in different groups through the Western Blot method, where β-actin and β-Tubulin are used as reference controls for protein immunoblotting hybridization (SLC7A11 and GPX4). FIG. 6B and FIG. 6C show analysis results for quantitative analysis of GPX4 and SLC7A11. FIG. 6D shows results of quantitative analysis of MDA around the hematoma of different groups. FIG. 6E shows results of quantitative analysis of GSH around the hematoma of different groups. FIG. 6F and FIG. 6G show detection results of the expression levels of SLC7A11 and GPX4 m RNA around the hematoma of different groups by using a real-time quantitative polymerase chain reaction (qRT-PCR). The results show that the ICH+EXO group has significant advantages in perihematoma SLC7A11 and GPX4 expression, β-actin and β-Tubulin control, GPX4 and SLC7A11 quantification, and real-time quantitative polymerase chain reaction compared to the ICH group. In some aspects, the results of the ICH+EXO group at least comparable to, if not better than, the Sham group. The results of the ICH+EXO group show that the human plasma exosomes from the healthy young people have significant curative effect on the brain injury associated with ferroptosis after ICH and are statistically significant.

The above test results indicate that the human plasma exosomes from healthy young people are able to treat the brain injury associated with ferroptosis after ICH, especially in animals with the dose of 40-60 μg.

L

What is claimed is:

1. A method for preventing and treating brain injury associated with ferroptosis after intracerebral hemorrhage, comprising:
administering human plasma exosomes to a subject;
wherein the human plasma exosome is derived from plasma of healthy young people.

2. The method according to claim 1, wherein the healthy young people are between 18 and 25 years old.

3. The method according to claim 1, wherein the human plasma exosomes are extracted and separated from the plasma of the healthy young people.

4. The method according to claim 1, wherein a dose of human plasma exosomes administered to a mouse is 40-60 μg.

5. A pharmaceutical composition for preventing and treating brain injury associated with ferroptosis after intracerebral hemorrhage, comprising: human plasma exosomes and pharmaceutical excipients;
wherein the human plasma exosome is derived from plasma of healthy young people.

6. The pharmaceutical composition according to claim 5, wherein the healthy young people are between 18 and 25 years old.

7. The pharmaceutical composition according to claim 5, wherein the pharmaceutical composition is in a form of an injection.
